(19) United States
(12) Patent Application Publication
(10) Pub. No.: US 2015/0366459 A1
(43) Pub. Date: Dec. 24, 2015

(54) OBJECT INFORMATION ACQUIRING APPARATUS AND LASER APPARATUS USED THEREIN

(71) Applicant: CANON KABUSHIKI KAISHA, Tokyo (JP)

(72) Inventors: Minoru Ohkoba, Sagamihara-shi (JP); Shigeru Ichihara, Tokyo (JP)

(21) Appl. No.: 14/736,381

(22) Filed: Jun. 11, 2015

(30) Foreign Application Priority Data
Jun. 18, 2014 (JP) 2014-125527

(51) Int. Cl. A61B 5/00; H01S 5/024; H01S 3/107; H01S 3/16; H01S 3/092; H01S 5/06; H01S 3/11

(52) U.S. Cl. CPC A61B 5/0095; H01S 5/0615; H01S 5/024; H01S 5/06; H01S 3/11; H01S 3/1643; H01S 3/1636; H01S 3/092; H01S 3/1611; H01S 3/1633; H01S 3/1625; H01S 3/1623; H01S 3/107; A61B 5/7203

(57) ABSTRACT

Provided is an object information acquiring apparatus provided with: a laser light source; a detection unit that detects a portion of laser light emitted from the laser light source; a determination unit that determines whether or not abnormal emission is contained in the laser light, based on a detection result of the detection unit; a radiation unit that radiates the laser light onto an object; a reception unit that receives acoustic waves that propagate from the object, based on radiation of the laser light; an acquisition unit that acquires information relating to the object, based on a reception result of the reception unit; and a control unit that controls output of the laser light, based on a determination result of the determination unit.

### BACKGROUND OF THE INVENTION

1. Field of the Invention

The present invention relates to an object information acquiring apparatus and a laser apparatus used therein.

2. Description of the Related Art

Photoacoustic tomography systems (photoacoustic measurement systems) are being developed for medical use that use short-pulse oscillation lasers (S. Manohar, et al., “Region-of-interest breast studies using the Twente Photoacoustic Mammoscope (PAM)” Proc. of SPIE, Vol. 6437, 643702). Photoacoustic tomography (PAT) refers to a technique for forming images by irradiating a measurement segment with a pulsed laser for about several tens to several hundred nanoseconds, receiving the photoacoustic waves generated therein with a probe, and processing the resulting received signals. The use of PAT makes it possible to analyze body functions from spectrum measurements based on the absorption coefficients of body tissue.

In addition, lasers using Q switching are used to generate short-pulsed light used to measure acoustic waves. Q-switched oscillation refers to a technology for generating high-output, short-pulse laser light by controlling an indicator of resonator performance in the form of a Q value that is a function of the half-width of an oscillation pulse. Laser oscillation at that time is referred to as giant pulse oscillation. An apparatus has been proposed that acquires object information by emitting laser light onto an object by laser oscillation using Q switching in this manner (Japanese Patent Application Laid-open No. 2013-89680).

### SUMMARY OF THE INVENTION

However, abnormal emission referred to as prelasing occurs when the properties of an apparatus become unstable in a laser apparatus using Q switching. In addition, this prelasing occurs at a timing that is earlier than the inherently required oscillation timing of short-pulsed giant pulses. Thus, this prelasing has to be detected and reduced. If prelasing occurs in an object information acquiring apparatus, light propagates to body tissue prior to the timing of giant pulse oscillation. As a result, acoustic wave signals are generated from the body tissue. Consequently, these signals become noise when analyzing the acoustic wave signals and impair the obtaining of accurate biological information (object information). In addition, since variation occurs in the width per se of a single giant pulse, desired acoustic wave signals are unable to be obtained. Moreover, there is a strong correlation between the occurrence of prelasing and the ambient temperature of the laser apparatus. However, suppressing the occurrence of prelasing by controlling the temperature of the laser apparatus has the effect of, for example, increasing the size of the object information acquiring apparatus or increasing production cost.

With the foregoing in view, an object of the present invention is to provide an object information acquiring apparatus that reduces the effects of prelasing.

In order to achieve the above-mentioned object, the present invention provides an object information acquiring apparatus, comprising: a laser light source; a detector configured to detect a portion of laser light emitted from the laser light source; a determination unit configured to determine whether or not abnormal emission is contained in the laser light, based on a detection result of the detector; a irradiator configured to radiate the laser light onto an object, a receiver configured to receive acoustic waves that propagate from the object, based on radiation of the laser light; an acquisition unit configured to acquire information relating to the object, based on a reception result of the receiver, and a controller configured to control output of the laser light, based on a determination result of the determination unit.

The present invention also provides an apparatus, comprising: a laser light source; a detector configured to detect a portion of laser light emitted from the laser light source; a determination unit configured to determine whether or not abnormal emission is contained in the laser light, based on a detection result of the detector; and a controller configured to control the laser light source, based on a determination result of the determination unit.

The present invention also provides an apparatus, comprising: a laser light source; a detector that detects a portion of laser light emitted from the laser light source; a determination unit that determines whether or not abnormal emission is contained in the laser light, based on a detection result of the detector; and a controller that controls a temperature of the laser light source, based on a determination result of the determination unit.

As has been described above, according to the present invention, an object information acquiring apparatus can be provided that reduces the effects of prelasing.

Further features of the present invention will become apparent from the following description of exemplary embodiments with reference to the attached drawings.

### BRIEF DESCRIPTION OF THE DRAWINGS

FIG. 1A is a drawing showing Practical Example 1 of the object information acquiring apparatus of the present invention;

FIG. 1B is a drawing showing a laser light source according to Practical Example 1 of the present invention;

FIGS. 2A to 2D are graphs indicating the relationship among normal oscillation, giant pulse oscillation and preleasing;

FIG. 3 is a drawing indicating the positional relationships of elements of a laser light sensor in Practical Example 1;

FIGS. 4A and 4B are graphs indicating typical radiation dose acquisition results in Practical Example 1;

FIG. 5 is a drawing showing a laser light sensor of an object information acquiring apparatus according to Practical Example 2 of the present invention;

FIG. 6 is a drawing showing a laser light sensor of an object information acquiring apparatus according to Practical Example 3 of the present invention;

FIG. 7 is a drawing showing a laser light sensor according to Practical Example 4 of the present invention; and

FIG. 8 is a drawing showing an example of the comparative art with respect to the present invention.

### DESCRIPTION OF THE EMBODIMENTS

The following provides a detailed explanation of embodiments of the present invention while referring to the drawings. Furthermore, the same reference numbers are used to indicate the same constituents as a general rule, and explanations thereof are omitted. However, detailed calculation formulas or calculation procedures and the like described to follow are to be suitably altered according to the configuration and various conditions of the apparatus to which the present invention is applied, and are not intended to limit the scope of the present invention to the following descriptions.

The object information acquiring apparatus of the present invention includes an apparatus that receives acoustic waves generated within an object as a result of irradiating the object with light (electromagnetic waves) such as near infrared rays, and uses a photoacoustic effect whereby object information is acquired in the form of image data. In the case of an apparatus that uses photoacoustic effects, the object information that is acquired refers to the generation source distribution of acoustic waves generated by light radiation, initial acoustic pressure distribution in an object, optical energy absorption density distribution or absorption coefficient distribution derived from initial acoustic pressure distribution, or the concentration distribution of a substance that composes a tissue. The concentration distribution of a substance refers to, for example, oxygen saturation distribution, total hemoglobin concentration distribution or oxidized/reduced hemoglobin concentration distribution.

In addition, object information of a plurality of locations in the form of characteristic information may also be acquired as a two-dimensional or three-dimensional characteristics distribution. Characteristics distribution can be generated in the form of image data indicating characteristic information within an object. Acoustic waves as referred to in the present invention are typically ultrasonic waves, and include those referred to as sound waves or photoultrasonic waves. Acoustic waves generated by photoacoustic effects are referred to as photoacoustic waves or photoultrasonic waves. An acoustic detector (such as a probe) receives acoustic waves generated or reflected within an object.

First, total output is smaller in the case of actual prelasing emission in comparison with giant pulse emission. Consequently, time-based resolution and detection of prelasing alone requires the use of a sensor having extremely high sensitivity and time resolution, thereby resulting in the problem of the sensor becoming expensive. Moreover, the amount of time from prelasing oscillation to the generation of a Q switching off signal for giant pulse oscillation is extremely short at several nanoseconds to several tens of nanoseconds. Consequently, this amount of time is excessively short for realistically controlling giant pulse oscillation not to be carried out during that time, thereby making the realization thereof difficult.

### Practical Example 1

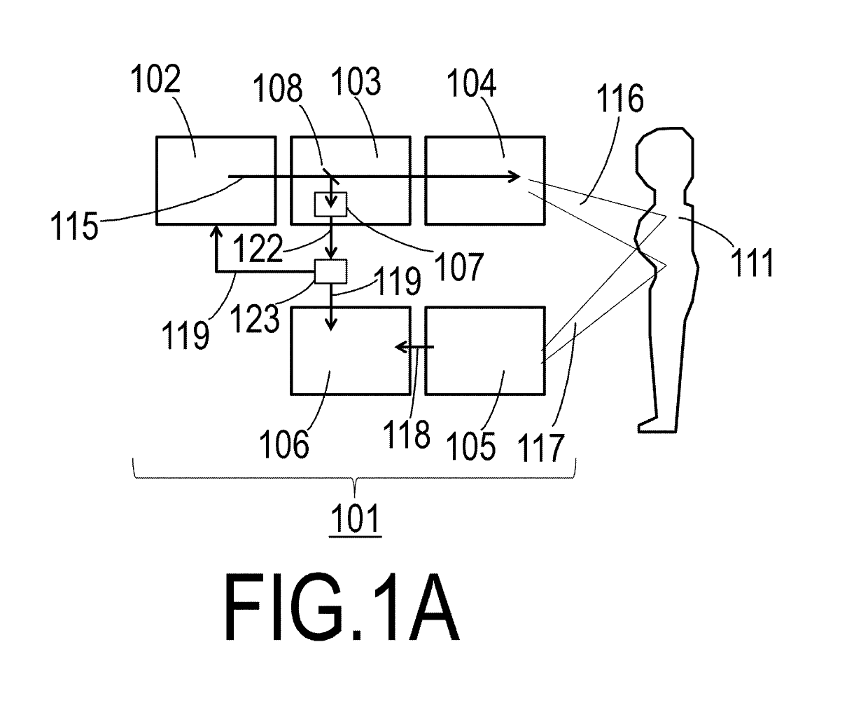

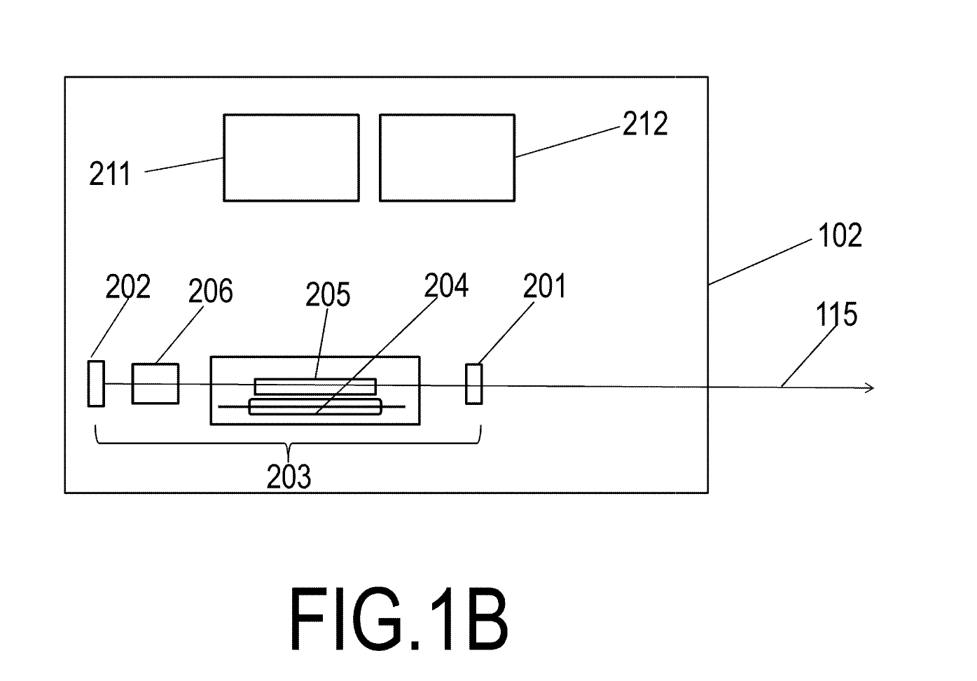

FIG. 1A is a drawing showing Practical Example 1 of the object information acquiring apparatus of the present invention. An object information acquiring apparatus 101 is provided with a laser light source 102. In addition, the object information acquiring apparatus 101 is further provided with a light-transmitting optical system 103, a irradiator in the form of a light-radiating optical system 104, an acoustic wave receiver 105 and an acoustic wave signal processing unit (an acoustic wave signal processor) 106. In addition, a detector in the form of a laser light sensor 107, a branch mirror 108, an object 111 and radiated light 116 are also shown in FIG. 1A. Moreover, an intensity detection signal 122, an acoustic wave signal 117, an electrical signal 118 and a prelasing determination signal, namely a determination result 119, are also shown in FIG. 1A.

The object information acquiring apparatus 101 is an apparatus that acquires information on the interior of the object 111 from a photoacoustic signal. A portion of the light energy that propagates through the interior of the object 111 is absorbed by an absorbing body (sound source) such as blood hemoglobin. Whereupon, the acoustic wave signal 117 is generated due to thermal expansion of the light-absorbing body, and that acoustic wave signal 117 propagates through the object interior. The propagating acoustic wave signal 117 is then converted to the electrical signal 118 with a probe in the acoustic wave receiver 105, and is transmitted to an acquisition unit (an acquisition device) in the form of the acoustic wave signal processing unit 106. The electrical signal 118 is converted to optical characteristic value distribution information and the like within an object by the acoustic wave signal processing unit 106 where it becomes object information. In addition to optical characteristic value distribution and absorption coefficient distribution, the generated object information can also include initial acoustic pressure distribution, substance concentration and oxygen saturation based thereon. Moreover, image data can also be included for forming and displaying an image (image reconstruction) based on this information.

The laser light source 102 supplies light for preferably passing through the object 111 in the form of a body and transmitting a photoacoustic signal attributable to a measurement target in the form of hemoglobin present in a blood vessel and the like. It is necessary for high-output light to propagate to the object 111 in order to enhance the signal accuracy of the photoacoustic signal, namely the acoustic wave signal 117. Laser light is used for this purpose. In addition, since it is necessary for the light to reach the measurement target in the form of hemoglobin in a blood vessel and the like with little absorption in the object 111, light for which there are limitations on the wavelength thereof and having wavelength characteristics of about 500 nm to 1200 nm is used in particular as light that easily propagates through the object 111. Consequently, an alexandrite laser or titanium-sapphire laser, which emits light of a wavelength within that range, is used preferably. In addition, pulsed light having a short pulse width, in which the pulse width is several tens to several hundred nanometers, is used for the laser light 115 in order to improve signal accuracy of the acoustic wave signal 117. A laser capable of giant pulse oscillation by Q switching is preferably used to generate such laser light having a high output and short pulse width. The laser light source 102 may be integrally incorporated in the object information acquiring apparatus 101 or may be installed outside thereof.

The light-transmitting optical system 103 has the function of propagating light from the laser light source 102 to the light-radiating optical system 104. Since the laser light source 102 and the light-radiating optical system 104 are at a distance from each other in terms of their arrangement and the laser light 115 ends up spreading, a lens, for example, is present in the optical path of the laser to suppress this spreading. In addition, in the case of not arranging the laser light source 102 and the light-radiating optical system 104 on a straight line in terms of their arrangement, a reflecting mirror and the like is arranged there between to adjust the direction of travel of the laser light 115 and guide the laser light to a desired location. Laser light may also be guided to a measurement apparatus, such as a timing trigger that measures the timing of light transmission required by the acoustic wave signal processing unit 106 or the laser light sensor 107 in the present invention, as necessary. Therefore, the branch mirror 108 is arranged in the optical path and that branched light is led to these measurement apparatuses. In addition, there are cases in which an optic fiber may be partially used for transmitting light within the light-transmitting optical system 103.

The light-radiating optical system 104 forms the radiated light 116 from the laser light 115 propagated by the light-transmitting optical system 103, and radiates the radiated light 116 onto a target measurement segment of the object 111. Consequently, the light-radiating optical system 104 fulfills the role of deforming the distribution of the amount of the laser light 115 to a preferable distribution of the amount of light for the object 111 primarily by spreading the laser light 115, for example. A lens or diffuser is arranged to form the radiated light 116 by preferably expanding and diffusing the laser light 115 so as to preferably obtain the acoustic wave signal 117 along with preventing the radiation dose to a body in the form of the object 111 from exceeding a specified value.

The acoustic wave receiver 105 has a probe that receives the acoustic wave signal 117. This probe that receives the acoustic wave signal 117 generated, for example, on the surface or in the interior of a body by pulsed light in the form of the radiated light 116, converts the acoustic wave signal 117 into the analog electrical signal 118. Any type of probe may be used provided it is able to receive acoustic wave signals, examples of which include probes using piezoelectric phenomena, probes using optical resonance and probes utilizing a change in electrostatic capacitance. The probe of the present embodiment has a plurality of receiving elements (such as piezo elements) arranged one-dimensionally or two-dimensionally, and the receiving elements are arranged in the shape of a spiral on the bottom of a bowl-shaped stationary component. The use of this multidimensional arrangement of receiving elements makes it possible to simultaneously receive the acoustic wave signal 117 at a plurality of locations, thereby making it possible to shorten measuring time. In the case of desiring to increase the number of locations where measurements are made with the probe, the probe may be made to receive the acoustic wave signal 117 by scanning a plurality of locations. After being converted to the electrical signal 118, the acoustic wave signal 117 received with the probe is used to generate characteristics information with the acoustic wave signal processing unit 106.

The acoustic wave signal processing unit 106 is composed of a computer or other information processing apparatus and circuitry, and carries out processing and calculations on the electrical signal 118. The acoustic wave signal processing unit 106 has a conversion unit such as an A/D converter that converts electrical signals obtained from the probe from analog signals to digital signals. The conversion unit is preferably able to process a plurality of signals simultaneously. This enables the amount of time until an image is formed (image reconstruction) to be shortened. The converted digital signals are stored in memory. The acoustic wave signal processing unit 106 generates object information such as optical characteristic value distribution by back projection in a time domain, for example, using the data and the like stored in memory.

FIG. 1B is a drawing showing the laser light source 102 of Practical Example 1 of the present invention. As shown in FIG. 1B, the laser light source 102 of the present invention is composed of a laser resonator 203, which is composed of two reflectors in the form of an output mirror 201 and a reflecting mirror 202, as well as a controller (a controlling unit) in the form of a laser controller 211 and a laser power supply 212. Furthermore, the wiring and the like of the laser controller 211 and the laser power supply 212 are omitted from the drawing. Here, the laser controller 211 is provided within the light source 102. Namely, the laser controller 211 is provided in front of the detector 107 provided in the light-transmitting optical system 103. The laser controller 211 can be provided with an information processing apparatus, such as a CPU, MPU or memory, and circuitry.

An excitation unit (an excitation device) 204, a laser medium 205 and a Q switch 206 are arranged within the resonator. Voltage applied to the excitation unit 204 and the Q switch 206 is controlled by the laser controller 211. In the case of using a flash lamp or semiconductor laser and using a rod-shaped laser medium 205, the excitation unit 204 carries out optical excitation from a side of the laser medium 205. A Pockels cell, which is an optical crystal of potassium dihydrogen phosphate (KDP) or dipotassium deuterium phosphate (DKDP) and the like, is used for the Q switch 206. A Pockels cell is an element that rotates the direction of polarization of light that passes through the element by using anisotropy to change refractive index in proportion to the strength of an electric field, and is widely used to obtain giant-pulsed light having a narrow oscillating pulse width and high output intensity. Although pulse width varies according to the type of laser medium, resonator length and resonator status, a pulse width of 100 ns or less is obtained. The configuration is as shown in FIG. 1B in the case of using an Nd:YAG crystal or alexandrite crystal. On the other hand, in the case of a titanium-sapphire laser, the second harmonic of the Nd:YAG laser serves as the excitation source of the titanium-sapphire crystal. In a titanium-sapphire laser, the present invention is applied to the Nd:YAG laser component serving as the excitation source. In the present description, an outline will be subsequently provided with reference to an alexandrite laser that excites the laser medium with a flash lamp. Alexandrite lasers have a gain in the range of 700 nm to 800 nm, and can be made to function as a variable wavelength laser by installing a wavelength selection mechanism composed of a birefringent filter between the laser medium 205 and the Pockels cell in the form of the Q switch 206 within the resonator.

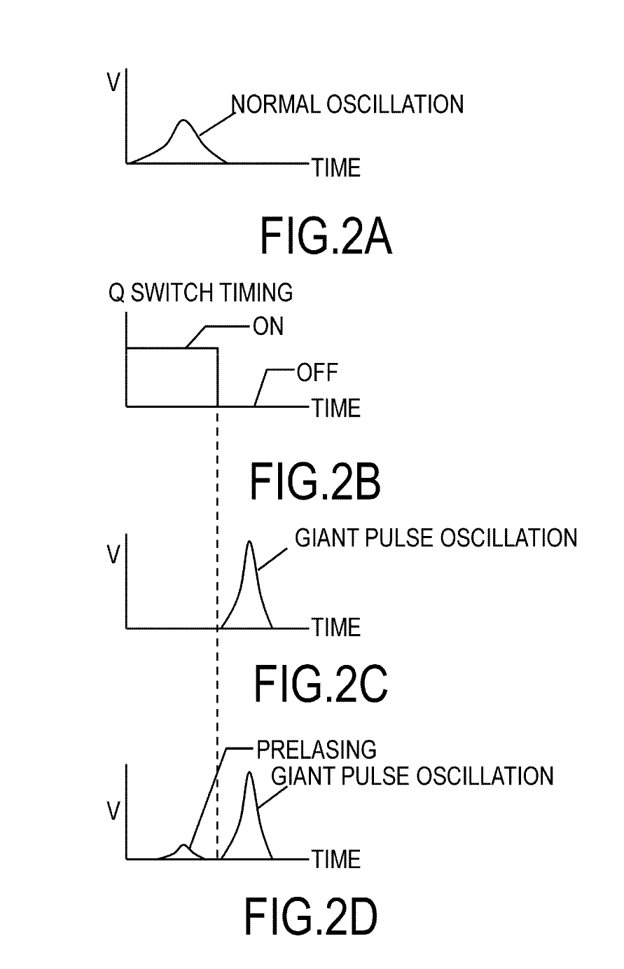

FIGS. 2A to 2D are conceptual drawings indicating the relationship among normal oscillation, giant pulse oscillation and prelasing. Here, an explanation of prelasing is provided using FIGS. 2A to 2D. Prelasing refers to a phenomenon that occurs when carrying out Q-switched oscillation. In the explanation of prelasing, an explanation is first provided of normal oscillation without using Q switching and giant pulse oscillation using conventional Q switching.

FIG. 2A indicates time-based changes in normal oscillation. During normal oscillation, a constant level of inverted distribution energy accumulates within the crystal due to excitation light, and when that energy has reached a threshold energy, laser light is generated from a resonator. In normal oscillation, pulse width is broader than giant pulse oscillation to be subsequently described.

FIG. 2B indicates time-based changes in on/off driving of Q switching. FIG. 2C indicates time-based changes in Q-switched oscillation. In the case of a laser that carries out Q-switched oscillation, a Q switch is arranged within the resonator, and oscillation is suppressed for a fixed period of time ranging from several tens of microseconds to several hundreds of microseconds by the Q switch. During that time, inverted distribution energy accumulates within the crystal due to excitation light, and a high level of energy is forcibly accumulated beyond the level of the threshold energy. After the fixed amount of time has elapsed, suppression of oscillation by the Q switch is canceled (the Q value of the resonator becomes higher), thereby resulting in generation of laser light having a high output and short pulse width. This is referred to as giant pulse oscillation.

FIG. 2D indicates time-based changes in overall oscillation in the case of the occurrence of prelasing. Prelasing oscillation refers to a phenomenon in which a portion of the accumulated energy escapes prior to giant pulse oscillation in a laser that carries out Q-switched oscillation. The causes of this phenomenon are diverse, including the configuration of members composing the Q switch and the optical characteristics of constituent members within the resonator. In addition, since Q-switched lasers are inherently designed for the purpose of realizing a mechanism that enables the accurate generation of giant pulses while suppressing prelasing, there are many cases in which the oscillation energy and pulse width of prelasing oscillation that has deviated from this objective are unstable. In the case of the occurrence of prelasing, prelasing occurs in a state in which Q switching is on during oscillation of a single pulse. Moreover, giant pulse oscillation also occurs thereafter when Q switching is off. Consequently, laser light ends up being generated which has a different pulse width from the case of accurate giant pulse oscillation.

In the case of solid-state lasers using a rod-shaped laser medium in particular, if prelasing oscillation occurs in the center of the rod where excitation efficiency is high, that oscillation takes on the form of seed light causing the subsequently occurring giant pulse oscillation to concentrate in the center. As a result, intense oscillation occurs having a characteristic intensity distribution to be subsequently described. Furthermore, the direction of polarization of reciprocal light can be changed using a device such as a Pockels cell for the Q switch that induces refractive index anisotropy in an electric field. Resonance is suppressed by this characteristic of Q switching. In the case of using such an optical shutter that suppresses resonance, the polarized state of giant pulse light differs from the polarized state of prelasing. This characteristic of Q switching is used in the examples to be subsequently described.

The laser light sensor 107 described in the present invention does not detect feint emission when the Q switch is on (resonance suppression period), but rather acquires oscillation intensity so as to include both feint light resulting from prelasing and giant pulse oscillation after Q switching, which emits feint light resulting from prelasing as seed light, has been switched off on a time axis. A signal corresponding to the detection result in the form of that acquired intensity is output to a determination unit 123 shown in FIG. 1A. The determination unit 123 is provided to distinguish emission attributable to prelasing from this output signal. As a result, detection of the occurrence of prelasing is realized even if the laser light sensor 107 does not have a very high level of time resolution. Furthermore, a “laser light sensor not having a very high level of time resolution” referred to here is a sensor that is only able to detect light intensity during the time width from the time prelasing occurs to the time giant pulse oscillation ends as shown in FIG. 2D, for example. Moreover, a “laser light sensor having high time resolution” is a sensor that is able to detect laser light intensity during the time width from the start of prelasing to the end of prelasing as shown in FIG. 2D.

The following provides an explanation of the method used to control an object information acquiring apparatus or laser apparatus contained therein in the case prelasing has occurred. Although there are cases in which a control method is introduced so as to reduce a suspected cause of prelasing provided the cause of prelasing can be presumed, there are also cases in which the object information acquiring apparatus or laser apparatus per se contained therein is shut down. In the case the cause is able to be presumed and is reversible, the member that composes the Q switch may be a member provided so as to change refractive index anisotropy by applying a voltage thereto in the manner of a Pockels cell. At this time, prelasing is presumed to occur as a result of the voltage deviating from the optimal applied voltage of the Pockels cell due to temperature effects and the like of the laser apparatus. In such cases, controlling the laser apparatus by changing the voltage applied to the Pockels cell makes it possible to suppress the occurrence of prelasing. A stable object information acquiring apparatus can be provided by providing such a control mechanism. In addition, in the case prelasing occurs randomly due to unstable operation of the Q switch, for example, identifying information as to whether or not emissions contain prelasing is output in combination with a photoacoustic signal. Use of only the acoustic wave signal obtained with emissions not containing prelasing, for example, for image reconstruction can then be used to remove noise from reconstructed images. Alternatively, information indicating that an image has been reconstructed based on laser light in which prelasing has occurred can also be output along with the reconstructed image.

Here, reference is again made to FIG. 1A. An aperture serving as a mode selector is arranged inside the resonator to enable the generation of pulsed light from the laser light source 102 that has a wavelength of 750 nm, pulse width of 100 nsec and repetition frequency of 20 Hz. In addition, ramped excitation, by which multimode pulsed light is generated having a beam profile diameter of 5 mm, and a Q-switched oscillation-type alexandrite laser light source, were used. Light was emitted at an output of 300 mJ per pulse. A convex lens having an F value of 1000 mm serving as the light-transmitting optical system 103 was arranged in the optical path to allow the laser light 115 to propagate in the form of nearly parallel light. A probe was arranged in an array for the acoustic wave reciever 105. The object 111 was taken to be a part of the body such as a woman's breast. The branch mirror 108 was used to branch the laser light 115 by arranging behind the light source 102 so as to demonstrate reflectance of 1% when reflecting at a 45° angle. The 1% of the laser light resulting after branching was guided to a laser light sensor 107a of the present invention. In addition, the laser system of the present example has an air-conditioning system in consideration of apparatus stability.

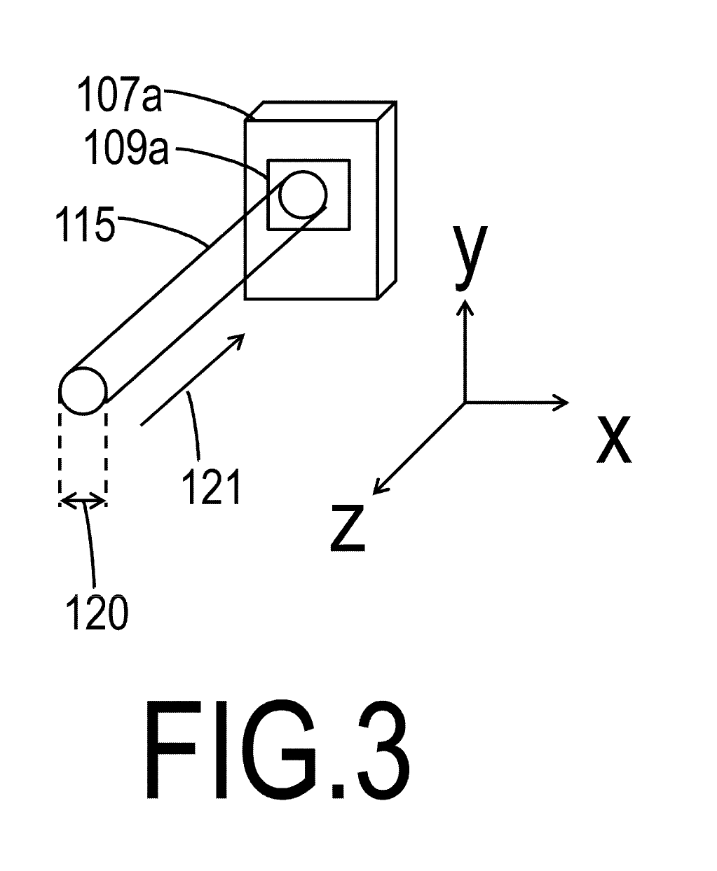

FIG. 3 is a drawing indicating the positional relationship of elements of a laser light sensor in Practical Example 1. The laser light sensor 107a used in the present example is explained using FIG. 3. FIG. 3 indicates the laser light sensor 107a, a photo acceptance unit 109a, the laser light 115, a laser light distribution width 120, and a laser traveling direction 121. The laser light sensor 107a used in the present example uses a beam profiler having the photo acceptance unit 109a of a size measuring 10 mm×10 mm. Moreover, there are 100 elements arranged at 10 mm intervals, and the photo acceptance unit 109a is arranged so as to lie in the direction of the xy plane that is perpendicular to the direction of the z axis when assuming the direction of the z axis to be the laser traveling direction 121. Namely, the photo acceptance unit 109a is, for example, an area sensor capable of measuring the distribution of laser light intensity in the xy plane. In addition, the photo acceptance unit 109a is arranged so as to lie in the center of the distribution of the laser light 115 in the center of the photo acceptance unit 109. As a result of adopting this layout, total irradiation energy (intensity) per pulse, including prelasing and giant pulse oscillation, for each 100×100 elements was acquired. The laser light sensor 107a then transmits the above-mentioned acquired results to the determination unit 123 of FIG. 1A. The transmission method may be wireless communication or using voltage or current signals by providing wiring.

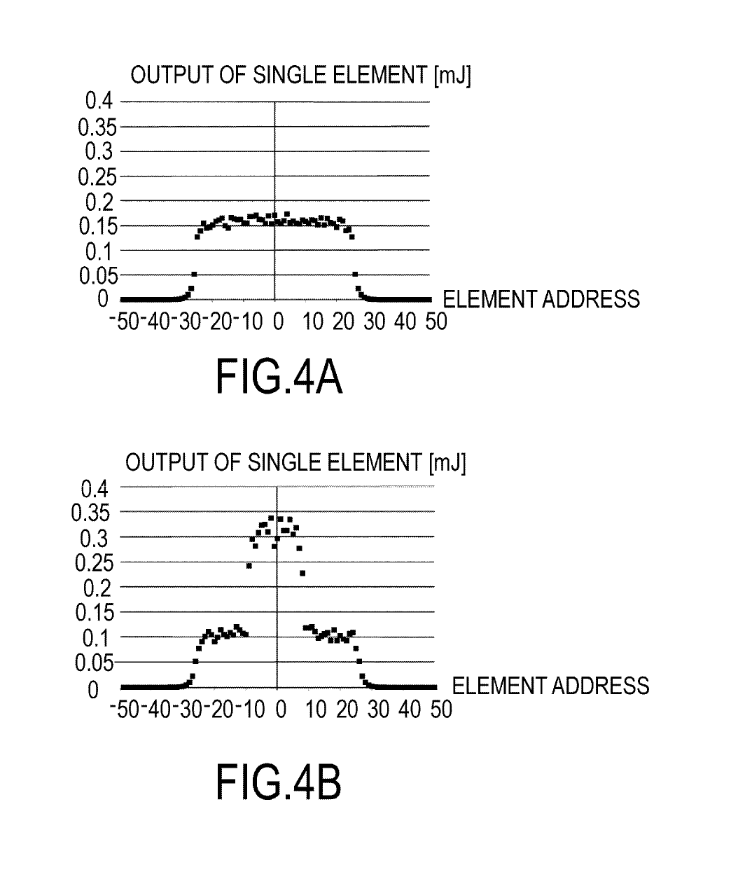

FIGS. 4A and 4B depict graphs indicating the results of acquiring typical radiation doses in Practical Example 1. In FIGS. 4A and 4B, the address (coordinates) in the x direction in FIG. 3 is plotted on the horizontal axis, and the energy of the laser light 115 that has entered a single element is plotted on the vertical axis. In addition, coordinate x=0 coincides with the center of the laser light distribution width 120 in FIG. 3. In addition, both FIGS. 4A and 4B indicate cross-sectional profiles that pass through the center of the beam profile with addresses in the y direction located in the center. FIG. 4A indicates the emission energy of typical giant pulse oscillation. FIG. 4B indicates emission energy when the combined emission energy of prelasing and giant pulse oscillation were acquired during the time that includes both the time when prelasing is occurring and the time during which giant pulse oscillation is occurring during a single pulse. The number of elements when the range over which giant pulse oscillation occurs has a diameter of 5 mm is equivalent to roughly 2000 elements, and roughly 0.15 mJ of energy per element is measured by the laser light sensor 107a.

Here, there is variation in the range of emission energy when the combined emission energy of prelasing and giant pulse oscillation is acquired. However, that energy is measured after concentrating in a range equivalent to a diameter of about 2 mm, which is narrower than the diameter of 5 mm of the range over which giant pulse oscillation occurs. The total amount of emission energy measured by each element remains at 300 mJ. Namely, this is the same as the total amount of emission energy of each element for the typical giant pulse oscillation shown in FIG. 4A. However, with respect to the range having a diameter of 2 mm where prelasing concentrates, output of 0.3 mJ per element is measured by the laser light sensor 107a. Namely, with respect to a diameter of 2 mm which is within this range, the amount of emission energy measured with each element when prelasing occurs shown in FIG. 4B is greater than the emission energy measured with each element shown in FIG. 4A. In addition, as is clear from FIG. 4B, a peak exists for the output of a single element when the element address is in the vicinity of zero.

Determination criteria were set in the determination unit 123 as to whether or not prelasing is occurring in a laser demonstrating such emission energy distribution characteristics. Namely, a determination threshold was set so that cases in which the average value of energy entering a range having a diameter of 2 mm is 0.25 mJ or more are determined to indicate the presence of prelasing. As a result, prelasing was able to be effectively detected. Namely, the determination unit 123 compares a prescribed value in the form of the above-mentioned determination threshold with the above-mentioned average value based on the results of detection by the laser light sensor 107a. As a result, when that comparison result is a result such that the above-mentioned average value exceeds the above-mentioned determination threshold, prelasing is determined to be occurring, and that determination result 119 is output. On the other hand, when that comparison result is a result such that the above-mentioned average value does not exceed the above-mentioned determination threshold, prelasing is determined not to be occurring, and that determination result 119 is output. Possible output destinations consist of the acoustic wave signal processing unit 106 and the laser light source 102.

Prelasing was able to be effectively detected by judging whether or not prelasing is occurring by monitoring the energy entering a diameter of 2 mm in a laser demonstrating such emission energy distribution characteristics. In addition, in the case prelasing has been determined to have occurred as a result of being detected, information was obtained indicating that the cause of the occurrence thereof is a rise in temperature of the laser system. Consequently, a control function was provided that suppresses the occurrence of prelasing by having the laser controller lower the set temperature of the laser system temperature control mechanism (such as an air-conditioner) by 0.1° C. As a result, an object information acquiring apparatus was able to be produced in which instability of giant pulse oscillation was suppressed. In addition, as a result of adopting a sensor configuration like that described above, the occurrence of prelasing was able to be detected easily even with the laser light sensor 107 in which time resolution is not that high.

### Practical Example 2

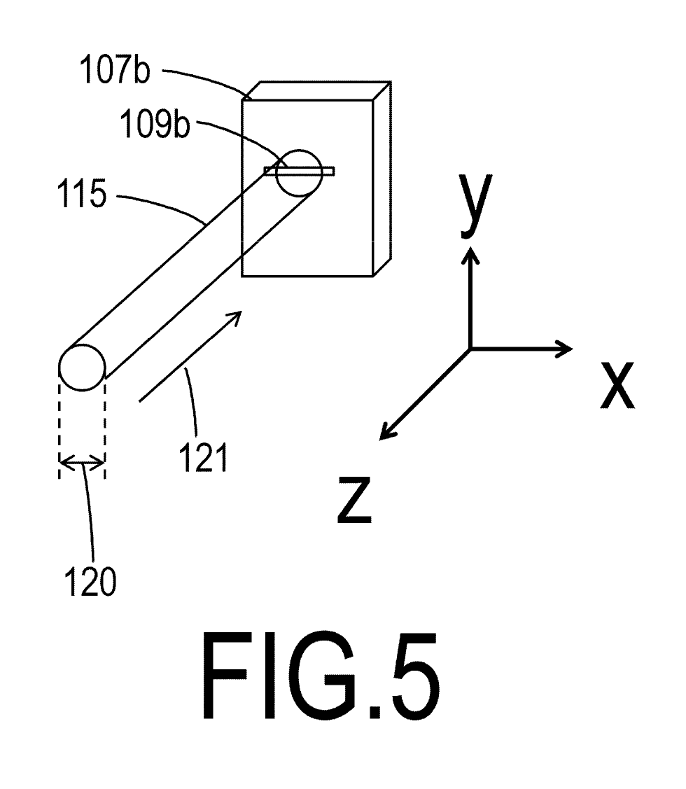

FIG. 5 is a drawing showing a laser light sensor of an object information acquiring apparatus according to Practical Example 2 of the present invention. The same reference numbers are used to indicate those constituents that are the same as those of Practical Example 1, and explanations thereof are omitted unless required. Namely, the laser light sensor of Practical Example 1 was an area sensor that detected the intensity distribution of laser light in the xy plane, namely in two dimensions. However, a one-dimensional line sensor 109b shown in this drawing may also be used for the photo acceptance unit 109b. Namely, the intensity of laser light on this line increases moving from the side near the periphery of the laser width 120 towards the side near the center of the laser width 120. Namely, the intensity distribution of laser light, which includes prelasing and giant pulse oscillation, can be acquired in this manner as well. In particular, the shape of the intensity distribution acquired by the line sensor 109b when the line sensor 109b is arranged so as to pass through the center of the laser width 120 is close to that shown in FIG. 4B. Accordingly, a configuration other than this sensor 107b can be composed in the same manner as Practical Example 1, and together with being able to provide an object information acquiring apparatus in which the effects of prelasing have been reduced, the number of photo acceptance elements can be reduced further than in the area sensor according to Practical Example 1, thereby leading to expectations of lower costs.

### Practical Example 3

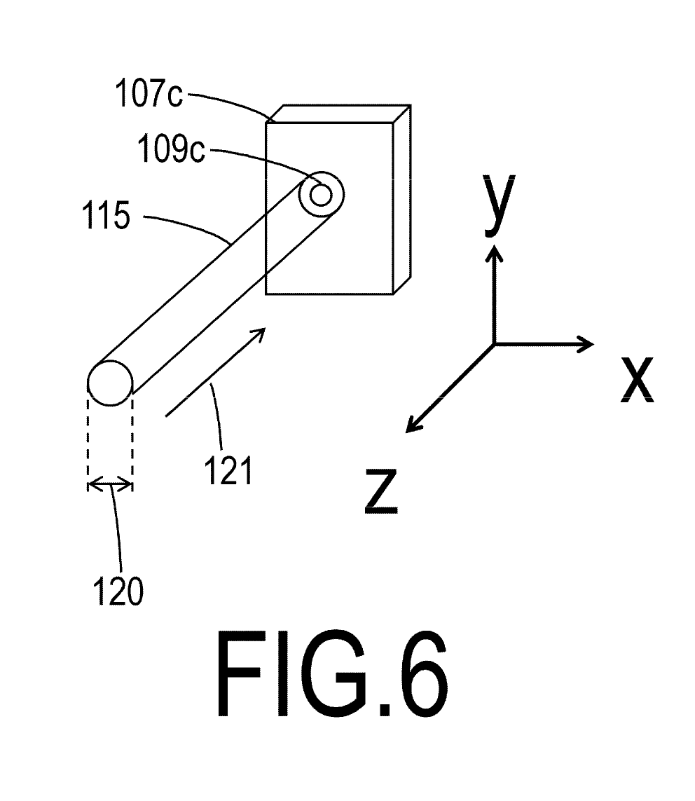

FIG. 6 is a drawing showing a laser light sensor of an object information acquiring apparatus according to Practical Example 3 of the present invention. The same reference numbers are used to indicate those constituents that are the same as those of Practical Example 1, and explanations thereof are omitted unless required. This laser light sensor 107c only detects laser light over a range that is smaller than the intensity distribution width of the laser light shown in FIGS. 4A and 4B. Namely, the intensity of laser light is detected over a range in which the element addresses of FIGS. 4A and 4B have a diameter of 2 mm. The shape of laser light sensor 109c of the present example differs from the laser light sensor in the form of the photo acceptance unit 109a of Practical Example 1. The element unit 109c of FIG. 6 is positioned in the center of the intensity distribution of the laser light 115 and the element unit 109c is not divided. The range covering a diameter of 2 mm is equivalent to about 310 of the elements of Practical Example 1. Consequently, measured values were about 310 times greater, energy in the case of the occurrence of giant pulse oscillation was 47 mJ and energy in the case of the occurrence of prelasing was 93 mJ. A mechanism by which the average value of this sensor 107c is output in combination with photoacoustic signal data is provided as feed-forward control. Furthermore, this laser light sensor 107c is, for example, a power meter having the element unit 109c that measures only the center by utilizing the fact that laser oscillation of a single pulse that occurs when prelasing has occurred is concentrated in the center of the laser light distribution width 120. The photo acceptance unit 109c has an aperture for measuring only the center. This aperture only allows passage of the laser light 115 that is in the vicinity of the center of the width 120, and in this case, within the range of a diameter of 2 mm. Namely, this range is the range where the laser light concentrates as previously described.

As a result of using the above-mentioned configuration, data containing abnormal emissions can be used while thinning out the data during image formation, thereby allowing the production of an object information acquiring apparatus that enables favorable data acquisition.

### Practical Example 4

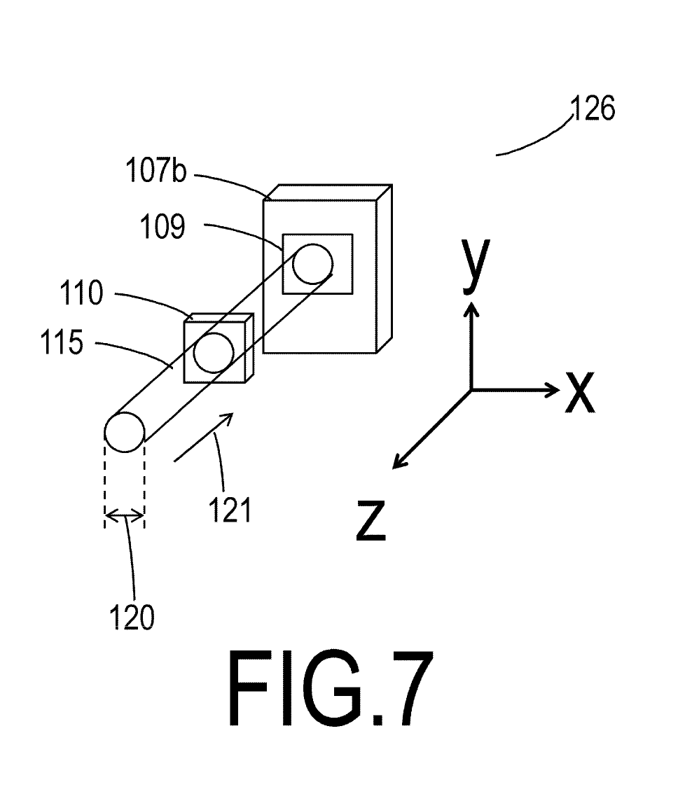

FIG. 7 is a drawing showing a portion of a laser light sensor according to Practical Example 4 of the present invention. The same reference numbers are used to indicate those constituents that are the same as those of the previously describe examples, and explanations thereof are omitted unless required. A laser light sensor unit 126 according to the present example is provided with the laser light sensor 107b of Practical Example 2 and a polarizing element in the form of a polarizing plate 110 provided on the front surface thereof, and is used to detect polarized light. Namely, this element unit 109 is an undivided single element sensor. The polarizing plate 110 is arranged in an orientation in which S-polarized light is strongly transmitted. Here, giant pulses constitute emission of P-polarized light. On the other hand, since prelasing light generated by this configuration is light for which oscillation is permitted only when Q switching is on, it is oscillated as S-polarized light. A mechanism whereby prelasing emissions that have passed through the polarizing plate 110 are received with this sensor 107b, and the energy value of prelasing emissions output as the reception result thereof is output in combination with photoacoustic signal data for each pulse, is provided as feed-forward control. Namely, the polarization characteristics of prelasing are utilized in the case of using an element such as a Q switch that uses electrical refractive index anisotropy in the manner of a Pockels cell. Namely, as shown in FIG. 7, the laser light sensor unit 126 identifies prelasing using the polarizing plate 110 in front of a power meter.

As a result, data based on abnormal oscillation can be used after being thinned out when forming an image from data that contains emissions attributable to abnormal oscillation, namely prelasing. Thus, an object information acquiring apparatus can be produced that uses an inexpensive laser light sensor 107b and is provided with the laser light sensor unit 126 that enables favorable data acquisition.

### Practical Example 5

Practical Example 5 was produced using the same constituent members as the object information acquiring apparatus used in Practical Example 1, and feedback control was carried out for control following determination of prelasing. More specifically, the occurrence of prelasing in this laser is characterized by a rise in temperature of the Q switch due to continuous use of the laser, and the voltage applied to the Q switch was determined to have a tendency to be below the lower limit voltage of the threshold value at which prelasing occurs. A set value of 2 kV was used for the voltage applied to the Q switch. Consequently, a feedback circuit was provided as a control function in the laser controller so that the voltage applied to the Q switch is raised by 100 V when prelasing is detected. As a result of providing this control function, an object information acquiring apparatus can be produced that easily detects prelasing and enables favorable data acquisition.

### Practical Example 6

Practical Example 6 was produced using the same constituent members as the object information acquiring apparatus used in Practical Example 1, and feed-forward control was carried out for control following determination of prelasing. More specifically, the occurrence of prelasing is detected based on the above-mentioned determination criteria by the determination unit 123 when prelasing has occurred. A mechanism, whereby information indicating that prelasing has occurred is output in combination with photoacoustic signal data for each pulse at that time, is provided as feed-forward control.

As a result, data containing abnormal emissions can be used while thinning out the data during image formation, thereby allowing the production of an object information acquiring apparatus that enables favorable data acquisition. Furthermore, image reconstruction may be carried out as is without the above-mentioned thinning processing by combining information indicating that prelasing has not occurred when prelasing has actually not occurred, and image reconstruction based on laser light in which prelasing has occurred may be notified to an operator.

### Modifications

Explanations of each of the examples are intended to be exemplary in terms of explaining the present invention, and the present invention can be carried out by suitably modifying or combining these examples within a range that does not deviate from the gist of the present invention. The above-mentioned processing and means of the present invention can be freely combined as desired provided they do not give rise to technical contradiction. Furthermore, the various characteristics of the present invention are not limited to the above-mentioned examples, and can be applied over a wide range. In addition, the object information acquiring apparatuses of the above-mentioned Practical Examples 1 to 6 can also be carried out using an information processing apparatus provided with a CPU or memory and the like that operates according to a program (software). Alternatively, each constituent of this object information acquiring apparatus may be composed with hardware such as circuity that enables input/output and arithmetic processing of information.

### Comparative Art

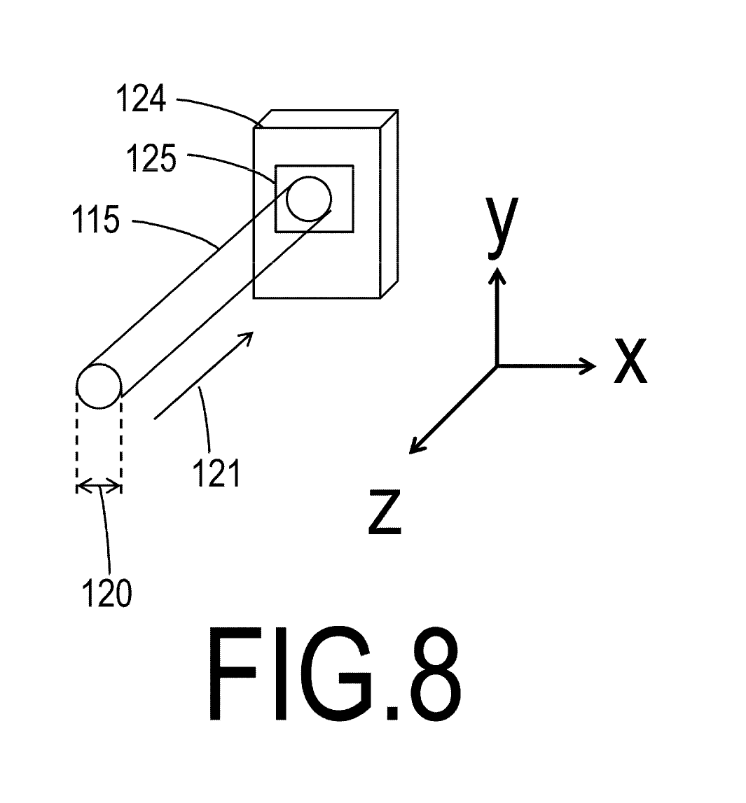

FIG. 8 is a drawing showing an example of the comparative art with respect to the present invention. The comparative art has the same configuration as Practical Example 1 with the exception of the laser light sensor used in Practical Example 1. An explanation is provided of a laser light sensor 124 used in the comparative art. Although an element unit 125 is provided at the same position as the element unit 109a shown in FIG. 3 of Practical Example 1, for example, it differs from the element unit 109a in that the element unit 125 is not divided and is a so-called single element sensor. The case is considered in which total energy is integrated during the time of a single pulse width. In this case, when prelasing occurs, the total amount of light energy resulting from combining both prelasing and giant pulse emission is not significantly different from the total amount of light energy during giant pulse emission alone when prelasing does not occur.

Consequently, whether or not prelasing has occurred cannot be determined with the single element sensor in the form of the laser light sensor 124 according to this comparative art. Consequently, an object information acquiring apparatus using this sensor of the comparative art was unable to acquire favorable data relating to object information. On the other hand, according to each of the examples of the present invention, an object information acquiring apparatus can be provided that is capable of detecting prelasing light, reducing the effects of prelasing and enabling the acquisition of favorable images as was previously described.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™) a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-125527, filed on Jun. 18, 2014, which is hereby incorporated by reference herein in its entirety.

What is claimed is:

1. An object information acquiring apparatus, comprising:
a laser light source;
a detector configured to detect a portion of laser light emitted from the laser light source;
a determination unit configured to determine whether or not abnormal emission is contained in the laser light, based on a detection result of the detector;
an irradiator configured to radiate the laser light onto an object;
a receiver configured to receive acoustic waves that propagate from the object, based on radiation of the laser light;
an acquisition unit configured to acquire information relating to the object, based on a reception result of the receiver; and
a controller configured to control output of the laser light, based on a determination result of the determination unit.

2. The object information acquiring apparatus according to claim 1, wherein the laser light source has a laser medium, two reflectors, a Q switch provided between the two reflectors and an excitation unit for optical excitation of the laser medium.

3. The object information acquiring apparatus according to claim 2, wherein the controller controls a Q value of the Q switch, based on the determination result.

4. The object information acquiring apparatus according to claim 2, wherein the controller controls a temperature of the Q switch, based on the determination result.

5. The object information acquiring apparatus according to claim 2, wherein the controller controls voltage applied to the Q switch, based on the determination result.

6. The object information acquiring apparatus according to claim 5, wherein the controller raises the voltage applied to the Q switch in a case where the determination unit has determined that abnormal emission is contained in detected light.

7. The object information acquiring apparatus according to claim 1, wherein the determination unit carries out the determination based on a comparison result obtained by comparing the detection result with a prescribed value.

8. The object information acquiring apparatus according to claim 7, wherein the prescribed value is a value based on an average value of intensity of the laser light within a range over which intensity of the laser light is distributed.

9. The object information acquiring apparatus according to claim 1, wherein the acquisition unit acquires information relating to the object, based on the determination result in addition to the reception result.

10. The object information acquiring apparatus according to claim 9, wherein the acquisition unit acquires information relating to the object, based on the reception result corresponding to the case where the determination unit determines that abnormal emission is not contained.

11. The object information acquiring apparatus according to claim 10, wherein the acquisition unit acquires information relating to the object without using the reception result corresponding to a case where the determination unit determines that abnormal emission is contained.

12. The object information acquiring apparatus according to claim 1, wherein the detector detects a peak in an intensity distribution of a portion of the laser light over a narrower range than a range over which the light intensity of a portion of the laser light is distributed.

13. The object information acquiring apparatus according to claim 1, wherein the detector is a line sensor or an area sensor.

14. The object information acquiring apparatus according to claim 1, wherein the detector detects light over a narrower range than a range over which light intensity of a portion of the laser light is distributed.

15. An apparatus, comprising:
a laser light source;
a detector configured to detect a portion of laser light emitted from the laser light source;
a determination unit configured to determine whether or not abnormal emission is contained in the laser light, based on a detection result of the detector; and
a controller configured to control the laser light source, based on a determination result of the determination unit.

16. The apparatus according to claim 15, wherein the laser light source has a laser medium, two reflectors, a Q switch provided between the two reflectors and an excitation unit for optical excitation of the laser medium.

17. The apparatus according to claim 16, wherein the controller controls a Q value of the Q switch, based on the determination result.

18. The apparatus according to claim 16, wherein the controller controls temperature of the Q switch, based on the determination result.

19. The apparatus according to claim 16, wherein the controller controls the voltage applied to the Q switch, based on the determination result.

20. The apparatus according to claim 19, wherein the controller raises the voltage applied to the Q switch in the case where the determination unit has determined that abnormal emission is contained in detected light.

21. An apparatus, comprising:
a laser light source;
a detector that detects a portion of laser light emitted from the laser light source;
a determination unit that determines whether or not abnormal emission is contained in the laser light, based on a detection result of the detector; and
a controller that controls a temperature of the laser light source, based on a determination result of the determination unit.

22. The apparatus according to claim 21, wherein the controller lowers the temperature of the laser light source in a case where the determination unit determines that abnormal emission is contained in detected light.
